(19) United States
(12) Patent Application Publication
(10) Pub. No.: US 2021/0352975 A1
(43) Pub. Date: Nov. 18, 2021

(54) SAFETY AIRBAG FOR HUMAN BODY PROTECTION

(71) Applicant: KUNSHAN ZHENGGONG MOULD CO., LTD., Kunshan (CN)

(72) Inventor: Longhai SONG, Kunshan (CN)

(21) Appl. No.: 16/466,021

(22) PCT Filed: Aug. 27, 2018
(86) PCT No.: PCT/CN2018/102456

§ 371 Date: Jun. 1, 2019

(30) Foreign Application Priority Data
Aug. 15, 2018 (CN) 201810926773.0

(51) Int. Cl. A41D 13/018

(52) U.S. Cl. CPC A41D 13/018

(57) ABSTRACT

The invention discloses a safety airbag comprising an upper airbag and a lower airbag. The upper airbag includes a back airbag and a head airbag laterally extending from a top of the back airbag. The lower airbag includes a waist airbag and a hip airbag extending downward from the waist airbag. The waist airbag can be folded, the hip airbag can be wound towards the waist airbag, and the waist airbag is superposed on the hip airbag. A fixing band is provided at each side of the hip airbag. After the low airbag is unfolded, the waist airbag is fitted to the waist, and the hip airbag protects the rear side and two sides of the hips and legs. The upper air inlet is in fluid communication with the lower air inlet, and the upper and lower airbags can be opened simultaneously to provide comprehensive protection for human body.

### FIELD OF THE INVENTION

The invention relates to a human body protection device, more particularly to a safety airbag for human body protection, which can provide protection when a human body falls down.

### DESCRIPTION OF THE RELATED ART

The technology of automobile airbag has been highly developed. The safety airbag is mainly composed of a sensor, a microprocessor, a gas generator, an airbag and the like. The sensor and microprocessor are used for determining the degree of collision, transmitting and sending signals. The gas generator ignites according to the signal indication, and the solid fuel is combusted to generate gas, such that the airbag is inflated rapidly. Meanwhile, the airbag is provided with a safety valve, when the airbag is overinflated or the pressure inside the airbag is greater than a certain value, some gas will be discharged automatically, thereby avoiding the damage to a person inside the vehicle due to crushing.

From the people-oriented view, accidental falling may cause damage to old people due to the weak body function, low physical quality and hip muscle atrophy. In severe cases, evenly it may be life-threatening. For the elderly people capable of disposing, if the electric induction device can determine the falling accidents within a few milliseconds at the moment of falling to the ground, the sensor commands the airbag to be opened by the electronic control device, and the inflated airbag can provide buffer protection for the worker's body, especially for loose bones and joints, thereby reducing the impact of falling on the elderly.

Generally the automobile airbag must be used with the safety belt; otherwise, the detonation of airbag will cause secondary damage to the driver and passenger. The automobile airbag is currently a passive device for safety protection on the cars, and can only be opened when an impact occurs.

The airbag device for human protection in prior art also has an airbag helmet. Compared with the ordinary helmet, the airbag is softer than the lining of the helmet and can absorb more impact force, but in use, it only can provide protection for the neck and head, and the other parts of the person are not protected. Thus, the protection area is small. When the user falls down, the other parts of the body such as the waist, the legs and the hips are subject to injury. Therefore, it is needed to develop an airbag protection device that is simple in structure, convenient for folding, and meet the theory of ergonomics.

### SUMMARY OF THE INVENTION

An object of the present invention is to overcome the above deficiencies in the prior art, and provide a safety airbag for human body protection, which can comprehensively protect a human body and improve the safety of daily activities of the elderly people.

In order to solve the above technical problems, the present invention provides a safety airbag for human body protection, including an upper airbag and a lower airbag, which provide protection for an upper part and a lower part of the human body respectively. The upper airbag includes a back airbag and a head airbag laterally extending from a top of the back airbag, such that the upper part of body such as a back, a neck and a head can be protected fully. The upper airbag is T-shaped after folding. The lower airbag includes a waist airbag and a hip airbag extending downward from the waist airbag to a knee. The waist airbag can be folded and the hip airbag can be wound towards the waist airbag, such that the waist airbag is superimposed on the hip airbag. The lower airbag has a waistband shape after folding. The safety airbag after folding occupies a small space, and is conveniently used in combination with a belt, a strap, and the like, and does not affect the daily appearance. A fixing band is provided at each side of a lower end of the hip airbag. The free ends of the fixing bands are connected with each other by a snap. After the lower airbag is unfolded, the waist airbag is fitted to protect the waist. The hip airbag surrounds the hips and the legs under the action of the fixing bands, to protect the rear side and two sides of the hips and the legs. The upper air inlet and the lower air inlet are in fluid communication with each other, and the upper airbag and the lower airbag can be inflated simultaneously. When working, the upper and lower airbags are simultaneously opened to protect the human body comprehensively.

Preferably, the back airbag is folded along a center line thereof. The head airbag is wound towards the back airbag, and the wound head airbag is folded towards the back airbag until the head airbag is completely folded in the length direction, so that the upper airbag is folded as an elongated structure, which is provided on the back of the human body, and thus the beauty of wearing of the human body will not be affected. Meanwhile, it is convenient to quickly open an upper storage device, thereby achieving rapid opening and protection of the human body.

Preferably, the inflation device is further connected with a trigger button and a gravity sensor. An inflating device is opened automatically after receiving a falling signal from the gravity sensor, or the inflating device is manually activated by means of the trigger button when a human body feels that falling is coming, thereby ensuring the safety airbag can be opened in various ways before damage is made to the human body.

Preferably, the head airbag has a thickness of 40-80 mm after inflation. The back airbag has a thickness of 80-100 mm, a length of 500-800 mm, and a width of 350-500 mm after inflation. The waist airbag and the hip airbag have a thickness of 40-80 mm after inflation. Thus, sufficient cushioning is provided, and the protection effect is ensured.

Preferably, the head airbag is U-shaped, and the head can be surrounded and protected completely.

Preferably, the length of the waist airbag is less than the length of the hip airbag, so that the hip airbag protects the legs as much as possible.

Preferably, the waist airbag has an arc-shaped structure fitted to the human body, and the hip airbag includes a plurality of columnar airbags, so that the lower airbag forms a skirt structure after inflation, thereby expanding the protection range of the lower body.

Preferably, at least one upper air inlet and at least one lower air inlet are provided, and the upper airbag and the lower airbag can be simultaneously inflated.

Preferably, two upper air inlets and two lower air inlets are provided, so that the inflation devices are arranged conveniently, and the inflation devices are located at two sides of the two upper air inlets and the two lower air inlets respectively.

The safety airbag for human body protection of the invention can comprehensively protect the human body and improve the safety of the daily activities of the elderly people.

### BRIEF DESCRIPTION OF THE DRAWINGS

FIG. 1 is a front view of the safety airbag after inflation according to the present invention;

FIG. 2 is a top view of the upper airbag after first folding according to the present invention;

FIG. 3 is a side view of the upper airbag after folding according to the present invention;

FIG. 4 is a schematic view of the lower air bag after folding according to the present invention.

### DETAILED DESCRIPTION OF THE PREFERRED EMBODIMENTS

The invention will be further illustrated in more detail with reference to the accompanying drawings and embodiments. It is noted that, the following embodiments only are intended for purposes of illustration, but are not intended to limit the scope of the present invention.

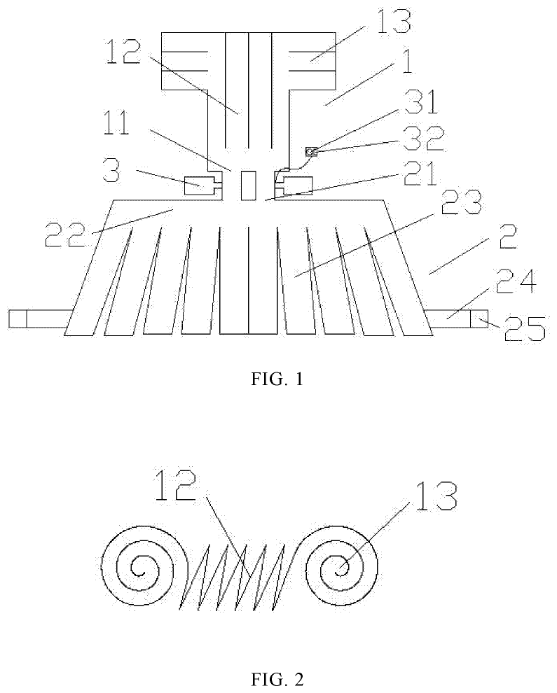

As shown in FIG. 1 and FIG. 4, a safety airbag for human body protection includes an upper airbag 1 and a lower airbag 2. The upper airbag 1 is used for protecting an upper part of the human body, and the lower airbag 2 is used for protecting the lower part of the human body, thereby providing full protection for the human body. The upper airbag 1 includes a back airbag 12 and a head airbag 13 extending laterally from back airbag 12, an upper air inlet is provided on a lower portion of the back airbag 12, the back airbag 12 protects the back and the vertebrae, and the head airbag 13 is extended laterally from the back airbag 12, such that not only the head but also the neck can be protected. In order to ensure that the head airbag 13 is further fitted to the head shape, the head airbag 13 laterally extending from the back airbag 12 is configured as U-shaped. The upper airbag 1 is T-shaped after folding. The lower airbag 2 includes a waist airbag 22 on a lower portion of which the lower air inlet 21 is provided, and a hip airbag 23 extending downward from the waist airbag 22 to a knee, thus, the lumbar bone, the hip bone, the sacrum, the tail bone and the femur are further protected. After the lower airbag 2 is unfolded, the waist airbag 22 is fitted to protect the waist, and the hip airbag 23 is opened outwards after inflation. The waist airbag 22 is folded, the hip airbag 23 is wound towards the waist airbag 22, and the waist airbag 22 is superposed on the hip airbag 23.The lower airbag 2 is folded as a waistband shape, and the safety airbag after folding occupies a small space and thus can be conveniently used with the belt and the strap, etc., and the daily appearance will not be affected. When inflating, the folded waist airbag 22 is rapidly inflated and unfolded, and the lower storage device of the lower airbag 2 is broken down at the fastest speed, then the hip airbag 23 and the fixing bands 24 are rapidly unwound and unfolded toward the hips, and the hips and hip bone are protected comprehensively. To ensure that the hip airbag 23 is fully fitted to the lower part of the human body, fixing bands 24 are connected at two sides of the lower end of the hip airbag 23, and the free ends of the fixing bands 24 are connected by a snap 25, and when the snap 25 is buckled up, the hip airbag 23 and the fixing bands 24 surround the legs. When folding, the fixing bands 24 are protruded from the lower airbag 2, and form a circle with the folded lower airbag 2. The hip airbag 23 is arc-shaped and surround the hips and legs, thereby providing full protection for the back and sides of the hips and legs. The upper air inlet 11 is in fluid communication with the lower air inlet 21, and the upper airbag 1 and the lower airbag 2 are simultaneously inflated by an inflation device 3, and the airbag can be quickly unfolded to provide comprehensive protection for the human body. In order to ensure the protection effect of the airbag, the head airbag 13 has a thickness of 40-80 mm after inflation, the back airbag 12 has a thickness of 80-100 mm, a length of 500-800 mm, and a width of 350-500 mm after inflation. The waist airbag 22 and the hip airbag 23 have a thickness of 40-80 mm after inflation. The length of the waist airbag 22 is less than the length of the hip airbag 23, and the hip airbag 23 is extended from the hips to the knee, thereby effectively protecting the hips and legs of the human body, to prevent the user from being injured after falling down. The waist airbag 22 has an arc-shaped structure fitted to the human body after inflation, and surround the waist. The hip airbag 23 includes a plurality of columnar airbags. Because the waist airbag 22 is arc-shaped, when inflated, the hip airbag 23 is expanded outwards to form a skirt structure, and is further bound by the fixing bands 24, thereby expanding the protection range.

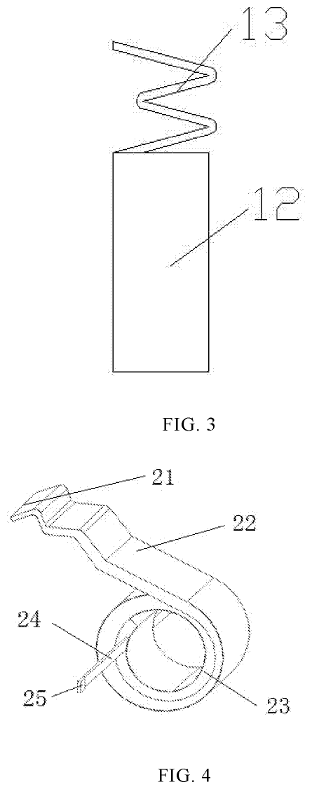

As shown in FIG. 2 and FIG. 3, when the upper airbag 1 is not inflated, the back airbag 12 is folded along a center line thereof, and the head airbag 13 is wound towards the back airbag 12, thereby the upper airbag 1 is completely folded in the vertical direction, and the wound head airbag 13 is downwards folded towards the back airbag 12 until the head airbag 13 is completely folded in the length direction, and the folded safety airbag is elongated and does not affect the appearance of wearing. When inflated, the air is introduced from the upper air inlet 11 on a lower portion of the back airbag 12, and the folded back airbag 12 and the head airbag 13 are rapidly inflated and unfolded to break down the upper storage device of the upper airbag 1 at the fastest speed. Then, the head airbag 13 is rapidly extended and spread laterally from head to fully wrap the head, thereby providing a comprehensive protection for the back, neck and head.

As shown in FIG. 1, the inflating device 3 is further connected with a trigger button 31 and a gravity sensor 32. The inflating device 3 is opened automatically upon receiving a signal of falling from the gravity sensor 32, or the inflating device 3 is manually activated by means of the trigger button 31 when a coming falling is felt by the human body. Thus, it is ensured that the safety airbag can be opened in various ways before the human body is injured.

In order to inflate the upper air bag 1 and the lower air bag 2, at least one upper air inlet 11 and at least one lower air inlet 21 are provided. In the embodiments of the invention, two upper air inlets 21 and two lower air inlets 21 are provided so that the inflation devices 3 are arranged conveniently. The inflation devices 3 are located at two sides of the two upper air inlets 11 and the lower air inlets 21, respectively.

The above description is only preferred embodiments of the present invention and not intended to limit the present invention, it should be noted that those of ordinary skill in the art can further make various modifications and variations without departing from the technical principles of the present invention, and these modifications and variations also should be considered to be within the scope of protection of the present invention.

What is claimed is:

1. A safety airbag for human body protection, comprising:
an upper airbag including a back airbag and a head airbag laterally extending from a top of the back airbag, wherein an upper air inlet is provided on a lower portion of the back airbag, and the upper airbag is T-shaped after folding; and
a lower airbag including a waist airbag and a hip airbag extending downward from the waist airbag to a knee, wherein a lower air inlet is provided on an upper portion of the waist airbag, the waist airbag is folding, the hip airbag can be wound towards the waist airbag, such that the waist airbag is superposed on the hip airbag, and the lower airbag has a waistband shape after folding, and
wherein the upper air inlet is in fluid communication with the lower air inlet, the upper air bag and the lower air bag are inflated simultaneously by a inflation device, and a fixing band is provided at each side of a lower end of the hip airbag, and free ends of the fixing bands are connected by a snap.

2. The safety airbag for human body protection as claimed in claim 1, wherein the back airbag is folded along a center line thereof, and the head airbag is wound towards the back airbag, and the wound head airbag is downwards folded towards the back airbag until the head airbag is completely folded in the length direction.

3. The safety airbag for human body protection as claimed in claim 1, wherein the inflation device is further connected with a trigger button and a gravity sensor.

4. The safety airbag for human body protection as claimed in claim 1, wherein the head airbag has a thickness of 40-80 mm after inflation, and the back airbag has a thickness of 80-100 mm, a length of 500-800 mm, and a width of 350-500 mm after inflation, the waist airbag and the hip airbag have a thickness of 40-80 mm after inflation.

5. The safety airbag for human body protection as claimed in claim 1, wherein the head airbag is U-shaped.

6. The safety airbag for human body protection as claimed in claim 1, wherein the length of the waist airbag is less than the length of the hip airbag.

7. The safety airbag for human body protection according to claim 1, wherein the waist airbag has an arc structure fitting with the human body, and the hip airbag comprises a plurality of columnar airbags.

8. The safety airbag for human body protection as claimed in claim 1, wherein at least one upper air inlet and at least one lower air inlet are provided.

9. The safety airbag for human body protection as claimed in claim 1, wherein two upper air inlets and two lower air inlets are provided.
